(19) United States
(12) Patent Application Publication
(10) Pub. No.: US 2026/0104400 A1
(43) Pub. Date: Apr. 16, 2026

(54) AIR QUALITY ASSESSMENT SYSTEM

(71) Applicants: Gabriel Keilholz, Bonnots Mill, MO (US); Amanda Keilholz, Bonnots Mill, MO (US)

(72) Inventors: Gabriel Keilholz, Bonnots Mill, MO (US); Amanda Keilholz, Bonnots Mill, MO (US)

(21) Appl. No.: 19/225,347

(22) Filed: Jun. 2, 2025

Related U.S. Application Data
(60) Provisional application No. 63/706,839, filed on Oct. 14, 2024.

(51) Int. Cl. G01N 33/00; G06T 19/00

(52) U.S. Cl. CPC G01N 33/0068; G06T 19/006

(57) ABSTRACT

An air quality assessment system is provided to assist an assessor in making an air opacity determination. A computer device has a display, a camera, and a controller in communication with the camera and the display. The display includes a first image and a second image, wherein the first image is a reality image of an area of interest where smoke is present and wherein the second image is an augmented reality image of the area of interest. The augmented reality image shows at least a portion of the area of interest at a particular percentage of opacity. An assessor can compare the reality image to the augmented reality image to aid the assessor in making an air opacity determination of the smoke in the area of interest or in the reality image. Optionally, the air quality assessment system can be used as a training and/or certification tool.

### PRIORITY

The present application claims the benefit of domestic priority based on U.S. Provisional Patent Application 63/706,839 filed on Oct. 14, 2024, the entirety of which is incorporated herein by reference.

### BACKGROUND

The assessment of air quality is an important part of maintaining healthy air. One such assessment is the determination of the opacity of air emissions. However, the determination of opacity is a difficult task that is fraught with inaccuracies, inconsistencies, and/or expense.

The practice of determining the opacity of air emissions is particularly important and impactful in a regulatory setting. Observations to determine the opacity of visible air emissions are used to assure compliance with air pollution laws and regulations. A visible emission observation is typically performed by a trained individual who makes a subjective determination of the opacity of air emissions based on their acquired knowledge and experience. Organizations must rely on these determinations as assurance they are in compliance with environmental laws at the risk of significant penalties if they are not. For example, a non-compliant facility can face fines up to $10,000 per day and/or legal action.

One specific method of determining the opacity of air emissions is US Environmental Protection Agency (EPA) Method 9—Visual Determination of the Opacity of Emissions from Stationary Sources. Method 9 is a visual test protocol that determines the opacity of air emissions by attempting to quantify by sight a level of opacity in a visible area of interest. The opacity of air emissions is the amount of light that is obscured by particulate matter in air emissions. 0% opacity is the lack of any visible air emissions and 100% opacity is the complete blockage of light by the air emissions. Method 9 is a statistical-based methodology that is used to determine the opacity of air emissions from sources such as smokestacks, dusty roads, process air vents and the like. An individual must be certified to perform Method 9 observations by attending a training event known as Smoke School. Smoke School uses a portable smoke generator that produces known opacities of black and white smoke. Individuals are trained on the smoke generator to recognize by sight various opacities of black and white smoke and then are tested on their ability to accurately assess fifty random smoke plumes produced by the smoke generator. When an individual passes the certification test, they are certified for six months and can perform opacity observations on any air pollution source. Facilities with the potential to produce air emissions are required by Federal, State and/or local regulations to comply with opacity limits. Individuals certified at Smoke School perform opacity observations to ensure the facility is in compliance, either in official or advisory capacities. Other countries have similar visible emissions standards and methodologies.

Though Smoke School has been used for decades and though certified individuals are highly skilled, the certification process and the application of Method 9 are highly subjective and determinations under Method 9 are prone to errors and/or inaccuracies. For example, smoke generators used to train and test students can vary in accuracy due to the physical interaction between the plume and ambient light, and weather conditions during smoke school are inconsistent and can translate to inaccurate smoke opacity and poor representation of real-world smoke behavior. In addition, certified individuals must rely on their visual memory to perform opacity observations up to six months after their smoke school training and certification event. This all combines to lead to a system where highly subjective observations are made and are done so in the absence of a record of the visual evidence. Furthermore, attending smoke school every six months is inconvenient for the individual seeking certification or re-certification, often forcing the individual to travel great distances and to take time out of their schedule. Since there can be high penalties for a facility's non-compliance with an air quality regulation, it is not ideal for the determinations to be based on a method that is subjective, without visual evidence, and/or without clearly repeatable accuracy between multiple observers.

Various attempts have been made to provide an alternative to in-person smoke school training for Method 9 determinations with mostly unsuccessful results. For example, LIDAR (light detection and ranging) equipment is a technology that uses lasers to measure the density of air emissions by illuminating particles and water vapor in the air. Historically, LIDAR has proven to be inaccurate when measuring low opacity levels and its size, complexity, and cost prevent it from being widely adopted. LIDAR also does not provide visual evidence that can be used to support opacity observations should the need arise. Attempts have also been made to remove the human-assessment element from the determination. For example, a Digital Opacity Camera System has been developed that allows a user to take a picture of smoke. The picture is analyzed by software to determine opacity. However, this method is difficult to use, the processing of the photos is slow, and results have not proven to be sufficiently accurate. In an effort to maintain the use of a human subjectivity while reducing the inconveniences of smoke school, efforts have also been made to create a virtual reality Smoke School in which students become certified by training and testing on a virtual reality headset. While useful in some instances, a certified individual must still rely on visual memory when performing opacity observations in the field, and this methodology still provides no visual evidence and record of the observation and assessment.

Therefore, there is a need for an improved air quality assessment system. There is a further need for an improved air opacity determination system. There is a further need for an air opacity determination system that utilizes subjective determinations in a more accurate and/or consistent manner. There is a further need for an air opacity determination system that utilizes an assessor's determination with reduced reliance on the assessor's visual memory. There is a further need for an air opacity determination system that enables faster and/or more accurate computerized determination of air opacity. There is a further need for an air opacity determination system that produces recorded evidence of a visual determination.

### SUMMARY

The present invention satisfies one or more of these needs. In one aspect of the invention, an improved air quality assessment system is provided.

In another aspect of the invention, an air quality assessment system in the form of an air opacity determination system is provided.

In another aspect of the invention, an air quality assessment system in the form of an air opacity determination system is provided to aid an assessor in making a determination of air opacity in an area of interest.

In another aspect of the invention, an air quality assessment system in the form of an air opacity determination system is provided to aid an assessor in making a determination of air opacity in an area of interest where emitted smoke is present.

In another aspect of the invention, an air quality assessment system in the form of an air opacity determination system is provided to aid an assessor in making a determination of air opacity in an area of interest by presenting to the assessor an augmented reality image of the area of interest.

In another aspect of the invention, an air quality assessment system in the form of an air opacity determination system is provided to aid an assessor in making a determination of air opacity in an area of interest by presenting to the assessor an augmented reality image of the area of interest, wherein the augmented reality image shows an image augmented or obscured to a particular opacity.

In another aspect of the invention, an air quality assessment system in the form of an air opacity determination system is provided to aid an assessor in making a determination of air opacity in an area of interest by presenting to the assessor an augmented reality image of the area of interest, wherein the augmented reality image shows an image augmented or obscured to a particular opacity, wherein the particular opacity is adjustable.

In another aspect of the invention, an air quality assessment system in the form of an air opacity determination system is provided to aid an assessor in making a determination of air opacity in an area of interest by presenting to the assessor an augmented reality image of the area of interest, wherein the augmented reality image shows an image augmented or obscured to a first particular opacity and an image augmented or obscured to a second particular opacity.

In another aspect of the invention, an air quality assessment system in the form of an air opacity determination system is provided to aid an assessor in making a determination of air opacity in an area of interest by presenting to the assessor an augmented reality image of the area of interest, wherein the augmented reality image shows an image augmented or obscured to a particular opacity, and wherein the augmented reality image is shown in juxtaposition to a reality image of the area of interest.

In another aspect of the invention, an air quality assessment system in the form of an air opacity determination system is provided to aid an assessor in making a determination of air opacity in an area of interest by presenting to the assessor an augmented reality image of the area of interest, wherein the augmented reality image shows an image augmented or obscured to a particular opacity, and wherein the augmented reality image is shown in juxtaposition to a reality image of the area of interest by being in a side by side relationship.

In another aspect of the invention, an air quality assessment system in the form of an air opacity determination system is provided to aid an assessor in making a determination of air opacity in an area of interest by presenting to the assessor an augmented reality image of the area of interest, wherein the augmented reality image shows an image augmented or obscured to a particular opacity, and wherein the augmented reality image is shown in juxtaposition to a reality image of the area of interest by being superimposed over the reality image.

In another aspect of the invention, an air quality assessment system in the form of an air opacity determination system is provided to aid an assessor in making a determination of air opacity in an area of interest by presenting to the assessor an augmented reality image of the area of interest, wherein the augmented reality image shows an image augmented or obscured to a particular opacity, and wherein the augmented reality image is shown in juxtaposition to a reality image of the area of interest, wherein the augmented reality image is adjustable relative to the reality image.

In another aspect of the invention, an air quality assessment system in the form of an air opacity determination system is provided to aid an assessor in making a determination of air opacity in an area of interest by presenting to the assessor an augmented reality image of the area of interest, wherein the augmented reality image shows an image augmented or obscured to a particular opacity, and wherein the augmented reality image is shown in juxtaposition to a reality image of the area of interest wherein the augmented reality image is adjustable in one or more of (i) position, (ii) orientation, (iii) size, (iv) color, and (v) opacity level.

In another aspect of the invention, an air quality assessment system in the form of an air opacity determination system is provided to aid an assessor in making a determination of air opacity in an area of interest by presenting to the assessor an augmented reality image of the area of interest, wherein the assessor is a human making a subjective determination.

In another aspect of the invention, an air quality assessment system in the form of an air opacity determination system is provided to aid an assessor in making a determination of air opacity in an area of interest by presenting to the assessor an augmented reality image of the area of interest, wherein the assessor is a trained artificial intelligence module that makes an opacity determination based on a comparison of the augmented reality image with a reality image.

In another aspect of the invention, an air quality assessment system in the form of an air opacity determination system is provided to aid an assessor in making a determination of air opacity in an area of interest by presenting to the assessor an augmented reality image of the area of interest, wherein the assessor is a combination of a human making a subjective determination and a trained artificial intelligence module that makes an opacity determination based on a comparison of the augmented reality image with a reality image.

In another aspect of the invention, an air quality assessment system in the form of an air opacity determination system is provided to aid an assessor in making a determination of air opacity in an area of interest by presenting to the assessor an augmented reality image of the area of interest, wherein the air quality assessment system saves a record of data related to the determination of air opacity.

In another aspect of the invention, an air quality assessment system in the form of an air opacity determination system is provided to aid an assessor in making a determination of air opacity in an area of interest by presenting to the assessor an augmented reality image of the area of interest, wherein the air quality assessment system includes a training module.

In another aspect of the invention, an air quality assessment system in the form of an air opacity determination system is provided to aid an assessor in making a determination of air opacity in an area of interest by presenting to the assessor an augmented reality image of the area of interest, wherein the air quality assessment system includes a certification module.

In another aspect of the invention, an air quality assessment system in the form of an air opacity determination system is provided to aid an assessor in making a determination of air opacity in an area of interest by presenting to the assessor an augmented reality image of the area of interest, wherein the air quality assessment system utilizes human subjective determinations in a more accurate and/or consistent manner, utilizes an assessor's subjective determination with reduced reliance on the assessor's visual memory, and/or enables faster and/or more accurate computerized determination of air quality by being easily analyzable by artificial intelligence systems, machine leaning systems, and the like.

In another aspect of the invention, an air quality assessment system in the form of an air opacity determination system is provided to aid an assessor in making a determination of air opacity in an area of interest by presenting to the assessor an augmented reality image of the area of interest, wherein the air quality assessment system is a downloadable application in a portable electronic device, such as a smart phone and/or tablet computer.

In another aspect of the invention, an air quality assessment system in the form of an air opacity determination system is provided to aid an assessor in making a determination of air opacity in an area of interest by presenting to the assessor an augmented reality image of the area of interest, wherein the air quality assessment system is a downloadable application in a portable electronic device, such as a smart phone and/or tablet computer, wherein the application and/or the portable electronic device is capable of communicating data related to a determination of air opacity to an internet based server or cloud system.

In another aspect of the invention, a method of assessing air quality comprises providing an air quality assessment system as described herein and using the air quality assessment system as described herein.

In another aspect of the invention, an air quality assessment system to assist an assessor in making an air opacity determination comprises a computer device comprising a display adapted to display a visual image, a camera adapted to capture an electronic image of an area of interest, and a controller in communication with the camera and the display, the controller capable of generating an augmented reality version of the electronic image of the area of interest, wherein the visual image displayed by the display includes a first image and a second image, wherein the first image comprises a reality image of the area of interest, wherein the second image comprises an augmented reality image of the area of interest, wherein the augmented reality image shows at least a portion of the area of interest at a particular percentage of opacity, and wherein an assessor can compare the reality image to the augmented reality image to aid the assessor in making an air opacity determination of the reality image.

In another aspect of the invention, a method of assessing air quality by determining air opacity comprises providing an air quality assessment system comprising a computer device comprising a display adapted to display a visual image and a camera adapted to capture an electronic image of an area of interest; capturing an electronic image of the area of interest; displaying the electronic image as a first image, wherein the first image is a reality image of the area of interest; displaying a second image, wherein the second image is an augmented reality image, wherein the augmented reality image shows at least a portion of the area of interest at a particular percentage of opacity; comparing the reality image to the augmented reality image; and making a determination of the air opacity of the reality image.

In another aspect of the invention, a method of assessing air quality by determining air opacity comprises providing an air quality assessment system comprising a computer device comprising a display adapted to display a visual image and a camera adapted to capture an electronic image of an area of interest; and using the air quality assessment system to certify or re-certify an individual under an air opacity assessment method by: displaying the electronic image of an area of interest as a first image, wherein the first image is a reality image of the area of interest; displaying a second image, wherein the second image is an augmented reality image, wherein the augmented reality image shows at least a portion of the area of interest at a particular percentage of opacity; tasking the individual to compare the reality image to the augmented reality image to make a determination of the air opacity of the reality image; and assessing the accuracy of the determination.

### BRIEF DESCRIPTION OF THE DRAWINGS

These features, aspects, and advantages of the present invention will become better understood with regard to the following description, appended claims, and accompanying drawings which illustrate exemplary features of the invention. However, it is to be understood that each of the features can be used in the invention in general, not merely in the context of the particular drawings, and the invention includes any combination of these features, where:

FIG. 1 is a schematic diagram of an air quality assessment system of the invention;

FIG. 2 is a schematic representation of another version of an air quality assessment system of the invention;

FIG. 3A is a schematic representation of another version of an air quality assessment system of the invention in a first step of operation;

FIG. 3B is a schematic representation of the version of an air quality assessment system of FIG. 3A in a subsequent step of operation;

FIG. 3C is a schematic representation of the version of an air quality assessment system of FIG. 3A in a final step of operation;

FIG. 4A is a schematic representation of another version of an air quality assessment system of the invention in a first step of operation;

FIG. 4B is a schematic representation of the version of an air quality assessment system of FIG. 4A in a final step of operation;

FIG. 5 is a schematic representation of another version of an air quality assessment system of the invention in which an augmented image is superimposed over a reality image;

FIG. 6A is a schematic representation of another version of an air quality assessment system of the invention in which an augmented image is superimposed over a reality image;

FIG. 6B is a schematic representation of the version of an air quality assessment system of FIG. 6A with the augmented image at a different opacity level;

FIG. 7A is a schematic representation of another version of an air quality assessment system of the invention in which an augmented image of a first size is superimposed over a reality image;

FIG. 7B is a schematic representation of the air quality assessment system of FIG. 7A with the augmented image being a second size;

FIG. 7C is a schematic representation of the air quality assessment system of FIG. 7A with the augmented image being a third size;

FIG. 8A is a schematic representation of another version of an air quality assessment system of the invention in which an augmented image of a first color is superimposed over a reality image;

FIG. 8B is a schematic representation of the air quality assessment system of FIG. 8A with the augmented image being a second color;

FIG. 8C is a schematic representation of the air quality assessment system of FIG. 8A with the augmented image being a third color;

FIG. 9A is a schematic representatation of another version of an air quality assessment system of the invention in which an augmented image includes multiple augmented images;

FIG. 9B is a schematic representation of the air quality assessment system of FIG. 9A with the augmented image being positioned in a second position;

FIG. 10A is a schematic representation of another version of an air quality assessment system of the invention in which an augmented image includes multiple augmented images;

FIG. 10B is a schematic representation of the air quality assessment system of FIG. 10A with the augmented image being positioned in a second position;

FIG. 11 is a schematic representation of another version of an air quality assessment system of the invention in which an augmented image includes multiple augmented images;

FIG. 12 is a schematic diagram of another version of an air quality assessment system of the invention;

FIG. 13 is a screen shot of a display screen using a version of an air quality assessment system of the invention;

FIG. 14 is a screen shot of a display screen using a version of an air quality assessment system of the invention;

FIG. 15 is a schematic diagram of another version of an air quality assessment system of the invention;

FIG. 16 is a schematic diagram of another version of an air quality assessment system of the invention;

FIG. 17 is a schematic representation of another version of an air quality assessment system of the invention; and

FIG. 18 is a schematic representation of another version of an air quality assessment system of the invention.

### DESCRIPTION

The present invention relates to an air quality assessment system. In particular, the invention relates to an air quality assessment system in the form of an air opacity determination system that uses augmented reality as an aid for an air quality assessor. Although the invention is illustrated and described in the context of being useful for making Environmental Protection Agency Method 9 determinations, the present invention can be used in other ways, as would be readily apparent to those of ordinary skill in the art. Accordingly, the present invention should not be limited just to the examples and embodiments described herein.

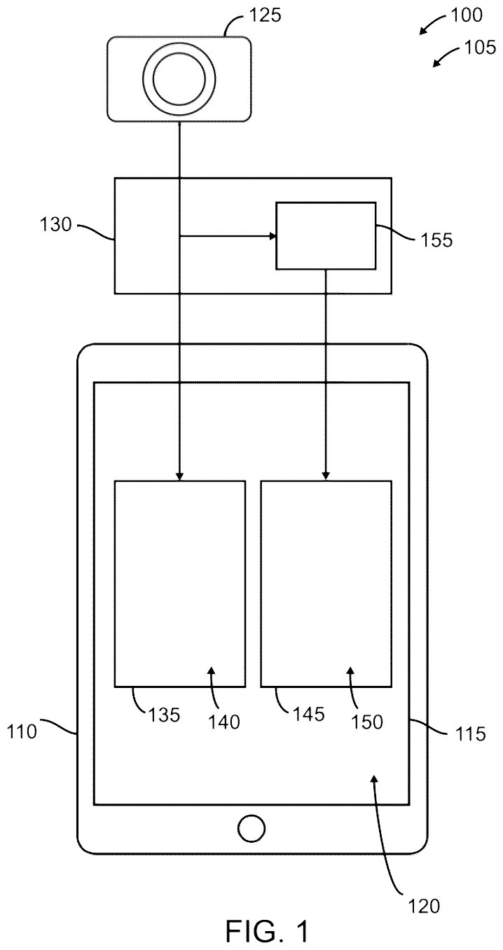

FIG. 1 shows a schematic representation of a version of an air quality assessment system 100 of the invention. The air quality assessment system 100 includes components that together enable and/or assist an assessor, such as a user of the system or other assessor, to make an assessment of air quality. In one version, such as the version of FIG. 1, the air quality assessment system 100 is a computer-based, electronic system 105 that enables and/or assists an assessor in making a determination of an air quality characteristic, such as the opacity of smoke that is being emitted into the air, as will be described. The components can be self-contained in a single device or can be separate components that are capable of being used and/or communicating with one another. The air quality assessment system 100 includes a display device 110 having a display, such as a display screen 115, that is capable of presenting a visual image 120 to an assessor, such as a user or an operator. A camera 125 is in electronic communication with the display device 110 though a controller 130. The controller 130 is adapted to receive an electronic signal from the camera 125 and generate a photographic and/or video image as part of the visual image 120 of the display screen related to the signal received from the camera 125. By image it is meant the image or graphical representation that is visualized or visualizable by a user or other assessor. The image is the image that is presented to the assessor whether the image be a single layer or multiple layers of electronic imagery. For example, when an electronic image is visible through a filter, the “image” would be the combination of the underlying image and the filter or the filtered image that is viewed by the user. By camera it is meant a single camera, a plurality of cameras, and/or a plurality of lens that operate collectively.

In use while performing an air quality assessment process, or prior to use, the camera 125 of the air quality assessment system 100 is aimed at an area of interest, such as an area where smoke is being emitted into the air so that an electronic or ‘digital image of the area of interest is captured by the camera 125. The visual image 120 of the air quality assessment system 100 includes a first image region 135 where a reality image 140 of the area of interest is displayed. The reality image 140 can be a photographic or video image of the area of interest, and the reality image 140 can be a live image or a recorded image. For example, the live image can be a live stream, a live video, or merely the image displayed when the camera is open. The reality image 140 is intended to capture and display a representation of the real visual appearance of the area of interest at a particular time or over a particular time period. An assessor then assesses the reality image 140 to make an assessment of air quality, such as by making a determination of an air quality characteristic, such as an opacity of smoke in the area of interest. The reality image is an electronic representation of reality or an electronic approximation of reality. For example, the reality image is not obscured or augmented in a manner that makes the image appear different than that way a scene appears or appeared in reality and/or different than other objects in a scene relative to their reality state.

The visual image 120 of the air quality assessment system 100 also includes a second image region 145 where an augmented reality image 150 of at least a portion of the area of interest is displayed. The signal from the camera 125 of the electronic image of the area of interest is passed within the controller 130 through an augmented reality module 155 that includes software or the like that has been programmed to, or is programmable to, augment the visual appearance of the area of interest in a desired manner and then causes the augmented image to be displayed as the augmented reality image 150 in the visual image 120 of the display device 110. By augmented reality image it is meant that at least a portion of the augmented reality image 150 is electronically altered in a manner that results in a visual presentation that is different from reality relative to the reality image 140. The augmented reality image 150 can be a portion of the reality image 140 that is augmented or can be a separate image that is augmented in a some way relative to the reality image 140. Said another way, the augmented reality image 150 is a portion of the display that is altered in a way that makes its displayed appearance less real than the displayed appearance of a reality image portion of the display. With the air quality assessment system 100 of FIG. 1, the augmented reality image 150 is designed to aid an assessor in making a determination of an air quality characteristic of the reality image 140, such as by allowing the assessor to compare the reality image 140 to one or more augmented reality images 150, as will be described. The assessor can be one or more users, such as one or more human operators, making a subjective determination, an electronic comparison system, or a combination of one or more user and an electronic comparison system, as will also be described.

The controller 130 can be any device capable of receiving input, performing calculations or executing code, performing calculations or executing code based on the input, producing an output signal, and/or producing an output signal as a result of the calculations or executed code. The controller 130 may be a single controller or multiple controllers that are capable of communication with one another within a single device or across multiple devices. The controller 130 may be in the form of a central processor that is capable of interacting with an operator via an input device, such as a keyboard, a graphical user interface, wireless communication, voice command, or any other manner. For example, the controller 130 may be embodied within personal computer, a laptop, a portable electronic device, such as a handheld device, a tablet computer, or a personal digital assistant, a server, a network of servers, a cloud network, or the like. One or more operators may interact with the controller 130 before, during, or after an air quality assessment process. The controller 130 can include various modules that allow it to perform calculations, algorithms, routines, and/or subroutines to process information, make determinations, and/or control the display of images. The controller 130 may further include modules, such as the augmented reality module 155, and/or one or more other modules, such as an input module, a storage module, a post-processing module, an artificial intelligence and/or machine learning module that use algorithms to parse data, learn from the data, and then to make determinations and/or predictions based on what was learned, a training module, and the like, as will be discussed.

The air quality assessment system 100 can be part of a dedicated device or can be in the form of a program or application that can be run on a computer system. For example, the air quality assessment system 100 can be in the form of a downloadable application that runs on a portable electronic device and that makes use of the camera, display screen, controller, and/or graphical user interface of the portable electronic device. The application can be capable of communicating data related to a determination of air opacity to an internet based server or cloud system.

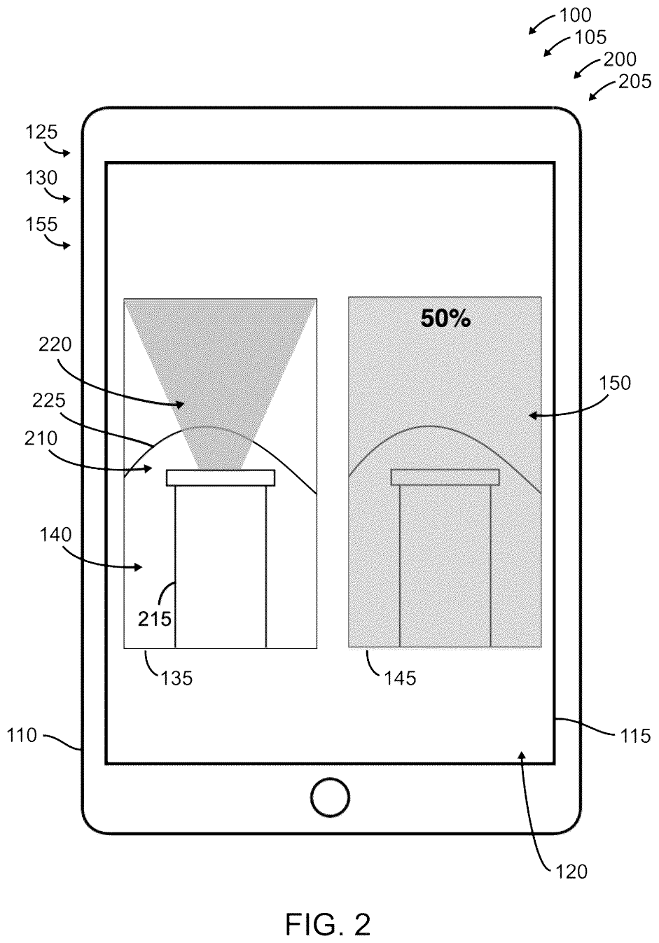

In one particular version, such as shown in FIG. 2, multiple components of the air quality assessment system 100 are embodied and/or included within a self-contained device 200. For example, the self-contained device can be a portable electronic device. The portable electronic device can be a handheld device, such as an Apple iPhone, a Google Pixel, a Samsung Galaxy, or other smartphone or the like, or a tablet, such as an Apple iPad, a Microsoft Surface Pro, Amazon Fire, or the like, with two or more of the camera 125, controller 130, and display screen 115 all incorporated into the device, in known manner. In such device the display screen 115 may include touch sensing technology that allows the display screen 115 to also serve as a graphical user interface so that an operator can input data to the controller 130 to control the visual image 120 or to otherwise make requests of the controller 130. In this version, the air quality assessment system 100 includes downloadable software, code, and/or subroutines that can be entered into and run by the controller 130 so that the controller 130 causes the display screen 115 to present one or more visual images 120 as described herein. For example, the software, code, and/or subroutines can be in the form of an app or an application that can be electronically installed into the controller 130. Alternatively, the air quality assessment system 100 can be preprogrammed and/or pre-installed into a dedicated device. In another version, a conventional handheld device or tablet can be in communication via the internet or other wireless communication with a server that houses the controller 130 and/or the augmented reality module 155. In this version, the reality image 140 or an electronic signal representative thereof is sent to the server where the reality image 140 is augmented into an augmented reality image 150, and the augmented reality image 150 is communicated back to the display screen 115 for presentation in the visual image 120.

FIG. 2 also shows a version of the invention in which the air quality assessment system 100 is an air opacity determination system 205. The determination of air opacity is an important way to make an air quality assessment. The air opacity determination system 205 is a tool that is useful in facilitating an assessor's determination of air opacity. With the air opacity determination system 205 a visual image 120 can be displayed by the display screen 115 that is designed to aid an assessor in making a determination of an opacity level of air, such as a determination of the opacity of smoke in the air. By smoke herein and throughout it is meant any region of air where a visible suspension, emulsion, obscuration, discoloration, or cloudiness of the air due to particles and/or gasses in the air is present, including for example visible dust, vapor, fire smoke, suspended particulate matter, fog, smog, fumes, exhaust and/or emissions into the air from a facility, a process, a reaction, a fire, and/or by natural occurrence. In the version shown, the reality image 140 is a picture or video of an area of interest 210, which in the version shown is a smokestack 215 that is or that is capable of emitting smoke 220. A background object 225 is shown behind the smoke 220. In the version shown, the background object 225 is a hillside. Alternatively, the background object 225 can be a building, sky, clouds, or any other object or combination. The opacity value is a percentage of opaqueness or lack of transparency through the smoke 220 in its most opaque region, which in the case of smokestack emissions is typically near the exit point of the smokestack 215. An assessor makes a determination of opacity by examining the degree to which the background object 225 is obscured by the opacity of the smoke 220. If the background object is completely obscured by the smoke, the opacity level is 100%, and if the smoke or lack thereof is completely transparent, the opacity level is 0%.

In the version of FIG. 2, the air opacity determination system 205 presents an augmented reality image 150 that can be used as a reference for the assessor as the assessor makes the determination of the opacity level of the smoke 220 in the reality image 140. For example, in the specific version of FIG. 2, the augmented reality image 150 is an image of at least a portion of the area of interest 210 augmented to represent what the background object 225 would look like at a particular percentage opacity level. In FIG. 2, the reference opacity level is shown as a particular percentage opacity of 50%. By comparing the appearances of the background object 225 in the reality image 140 and in the augmented reality image 150, the assessor can then easily determine whether or not the smoke 220 in the reality image has a higher, lower, or approximately equivalent opacity level as the reference augmented reality image 150. In the example shown, it can be seen that the reality image smoke 220 is more opaque than the reference 50% opacity, and the assessor can then clearly state that the opacity level is greater than 50% and can, if desired, estimate the opacity level to be, say, 70% to 80%.

The version of the air opacity determination system 205 of FIG. 2 can be particularly useful when it is the system is being used to make a quick determination of whether or not smoke opacity in an area of interest 210 exceeds a certain maximum opacity level. In this situation, the reference augmented reality image 150 can be set to show an opacity level that is at the maximum level. This enables the assessor to make a quick and easy determination as to whether or not the smoke 220 in the reality image 140 exceeds the maximum.

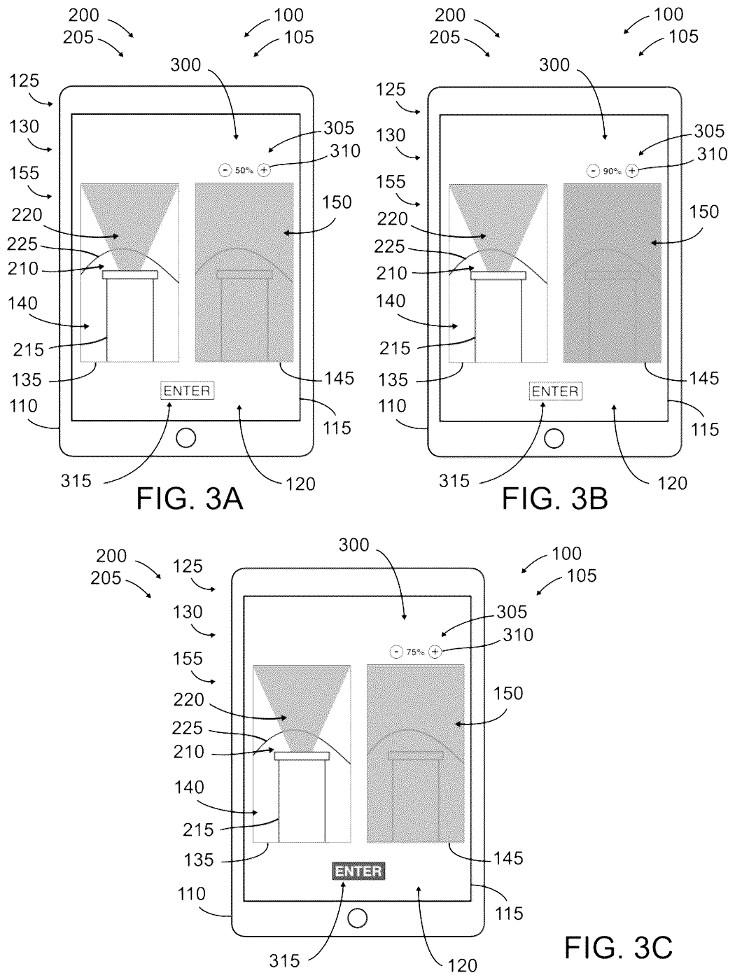

Another version of an air quality assessment system 100 in the form of an air opacity determination system 205 is shown in FIGS. 3A, 3B, and 3C. This version allows an assessor to make a more accurate determination of the opacity level of smoke 220 in the reality image 140 by including an augmented reality adjustment system 300 that makes the augmented reality image 150 adjustable in on the visual image 120. For example, the augmented reality image can be adjustable in in one or more of (i) position, (ii) orientation, (iii) size, (iv) color, and (v) opacity level. In the version of FIGS. 3A, 3B, and 3C, the simulated opacity level in the augmented reality image 150 is adjustable in terms of its opacity level by an opacity level adjuster 305, such as one or more opacity level control buttons 310, which can be touchscreen buttons on the display screen 115, mechanical buttons on the device 200, voice command electronic buttons, or the like. To use this version, an initial visual image 120, such as the one shown in FIG. 3A, is presented to the assessor. In the initial visual image 120, the augmented reality image 150 is shown at an initial level which can be set by the assessor or can be an automatically preset initial level. The assessor makes an initial determination as to whether the smoke 220 in the reality image 140 has higher or lower opacity than the augmented reality image 150. In the example shown, the opacity level is higher than the reference, and the assessor then adjusts the augmented reality image 150, such as by selecting a particular percentage of opacity of 90% opacity, as shown in FIG. 3B. The process continues until the assessor homes in on the closest simulation, as shown in FIG. 3C. The assessor then makes a determination that the opacity level of the smoke is the opacity level associated with the closest simulation, in this case 75%, and makes a note of such, either on a form or notebook or by contacting an opacity level enter button 315 on the device 200 that automatically enters and saves the determination. A screen shot can also be taken, or can be automatically taken with the depression of the opacity level enter button 315, to save the visual image 120 as evidence of the determination.

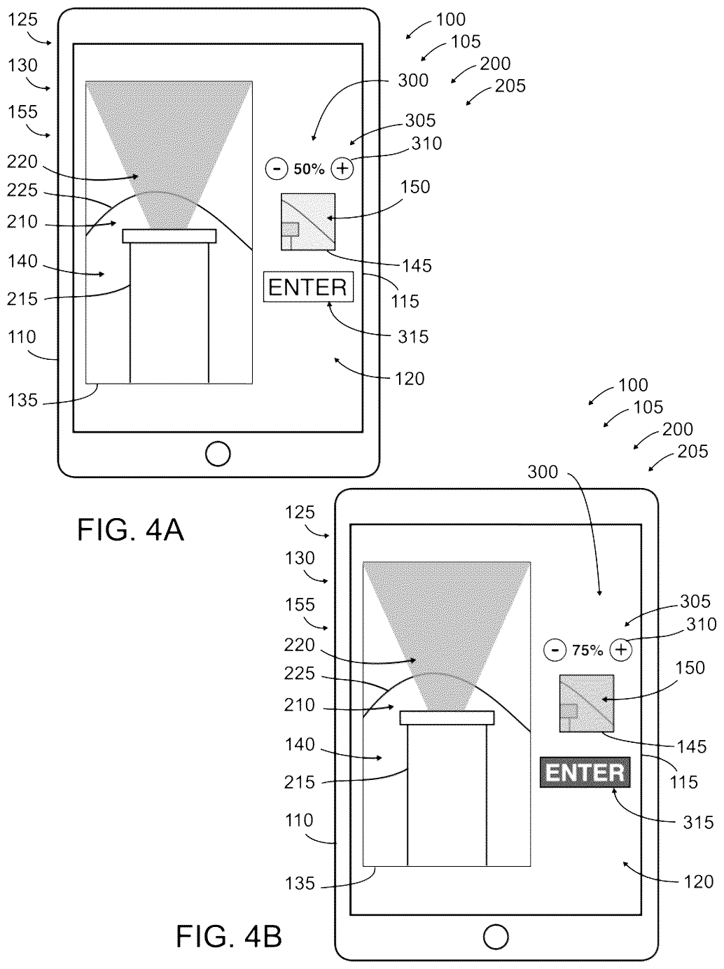

In the versions of FIG. 2 and FIGS. 3A, 3B, 3C the augmented reality image 150 shown does not include the smoke portion of the reality image 140. This can be accomplished in any of a number of ways. For example, smoke can be digitally removed from the image before it is augmented with the opacity simulation. In another version, the camera 125 can capture the image of the area of interest 210 before there is emitted smoke present. Alternatively, the assessor can merely ignore the portion of the augmented reality image 150 that includes the smoke. In yet another version, the portion of the augmented reality image 150 where the smoke is present can be cropped out so that the augmented reality image 150 only shows a portion of the simulated reality. A version of an air quality assessment system 100 with a reduced field augmented reality image 150 is shown in FIGS. 4A and 4B. By showing only the most relevant portion of the augmented reality image 150, there are fewer distractions from artificial opacity from the combination of smoke and augmented opacity. In addition, the reduction in size of the augmented reality image 150 provides more available space on the display screen 115 for enlarging the reality image 140 to help make the determination more accurate or for other features.

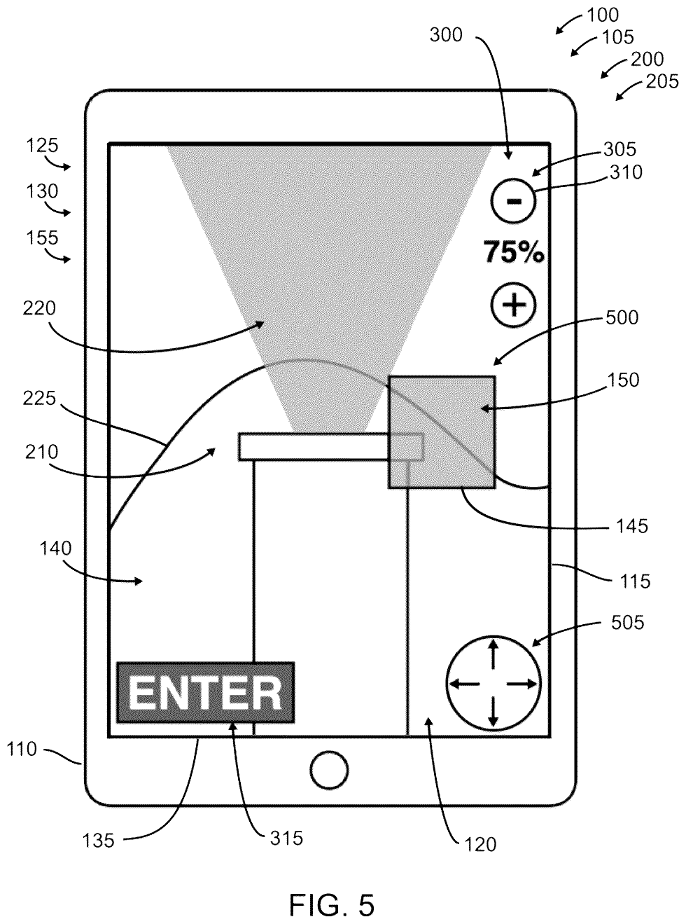

FIG. 5 shows another version of an air quality assessment system 100 with air opacity determination system 205. In the version of FIG. 5, the visual image 120 includes a superimposed display 500 where the augmented reality image 150 is superimposed over the reality image 140 to further aid the determination of opacity levels. This version allows the reality image 140 to be even larger and can occupy as much of the display screen as desired, including the entire display screen 115. In a particularly useful version, the augmented reality image 150 is moveable on or relative to the reality image 140 to a position on the reality image 140 that is most useful for an assessor making the opacity determination. Accordingly, in this version, the augmented reality adjustment system 100 comprises an image position system 505 which can be provided for controlling the position of the augmented reality image 150 on the reality image 140 and/or for controlling the positing of the reality image 140 relative to the augmented reality image 150. In the version illustrated, the image positioning system 505 is illustrated conceptually as directional buttons that can be depressed to move an image in a particular direction. Alternatively, an image can be moved by touching and dragging in conventional touchscreen manner.

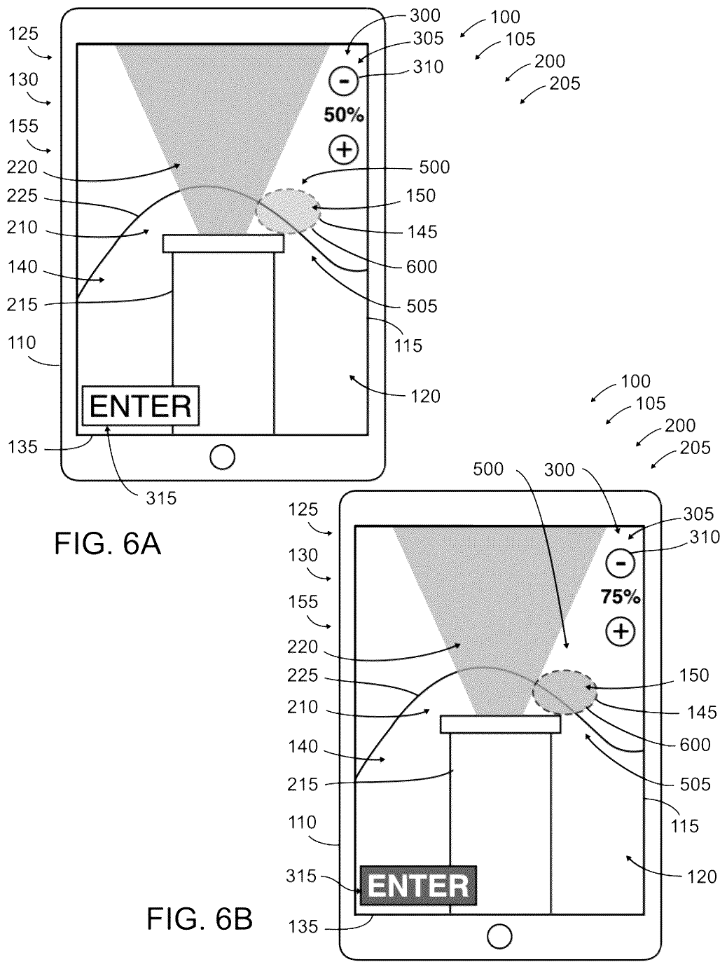

FIGS. 6A and 6B show another version of an air opacity determination system 100 with a superimposed display 500. The version of FIGS. 6A and 6B is similar to the version of FIG. 5, but with the version of FIGS. 6A and 6B, the augmented reality image 150 is in the form of an opacity augmentation bubble 600. The opacity augmentation bubble 600 is positionable at any location over the reality image 140 and is adapted to show what the area within the opacity augmentation bubble 600 would look like if obscured by a particular percentage of opacity, such as a predetermined or entered percentage of opacity. In the version of FIGS. 6A and 6B, the opacity level of the area within the opacity augmentation bubble 600 is adjustable by the opacity level adjuster 305 or the like. The opacity level is set at an initial value in FIG. 6A. As the assessor performs the assessment in this situation, it is easily determined that the initial opacity value is lower than the opacity of the smoke 220 in the reality image 140. Accordingly, the opacity level of the opacity augmentation bubble 600 is adjusted until it is at a level that corresponds with the opacity of the smoke 220 in the reality image 140, as shown in FIG. 6B. At this point, the determined opacity value can be entered by depressing the opacity level enter button 315 to save the determination and any desired additional information. Optionally, the opacity augmentation bubble 600 can be positionable on the reality image 140 by an image positioning system 505 of the augmented reality adjustment system 300. In the version of FIGS. 6A and 6B, the image position system 505 is a touch and drag system, but can alternatively be any other positioning system.

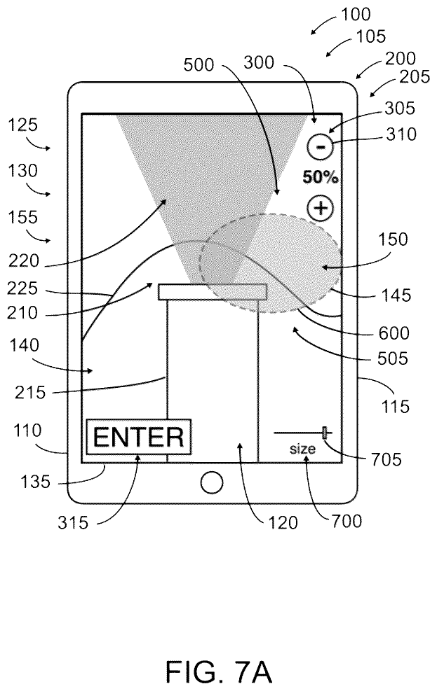

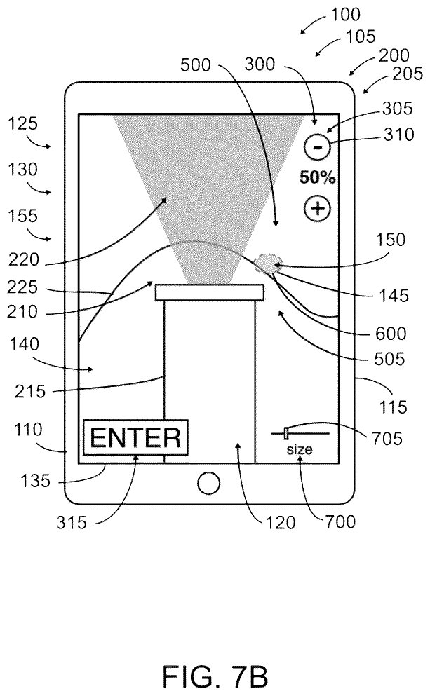

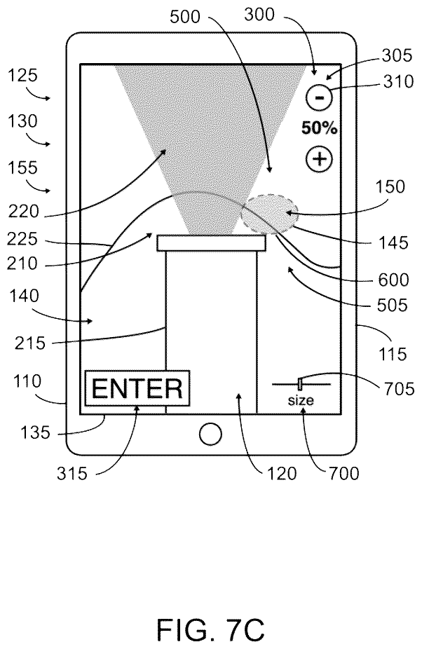

FIGS. 7A, 7B, and 7C show another version of an air opacity determination system 205 similar to the version of FIGS. 6A and 6B, but with the version of FIGS. 7A, 7B, and 7C, the size of the opacity augmentation bubble 600 is adjustable. Accordingly, in this version, the augmented reality adjustment system 300 includes a size adjustment system 700 instead of or in addition to the image positioning system 505. The size adjustment system 700 in the version of FIGS. 7A, 7B, and 7C is a sliding size scale 705 on the visual image 120 that a user can interact with the adjust the size of the opacity augmentation bubble 600. In FIG. 7A, the opacity augmentation bubble 600 is set to be very large on the reality image 140. In this particular case, the large size of the opacity augmentation bubble 600 obscures the smoke 220 and makes it difficult to make an accurate determination. In FIG. 7B, the size of the opacity augmentation bubble 600 is too small to be ideally effective. In FIG. 7C, the size is adjusted to be conveniently positioned next to the smoke 220 that is being assessed, and it is easy for the assessor to see that in this example case, the opacity level of the opacity augmentation bubble 600 is less than the opacity of the smoke 220 in the reality image 140, and the assessor knows to increase the opacity level setting of the opacity augmentation bubble 600.

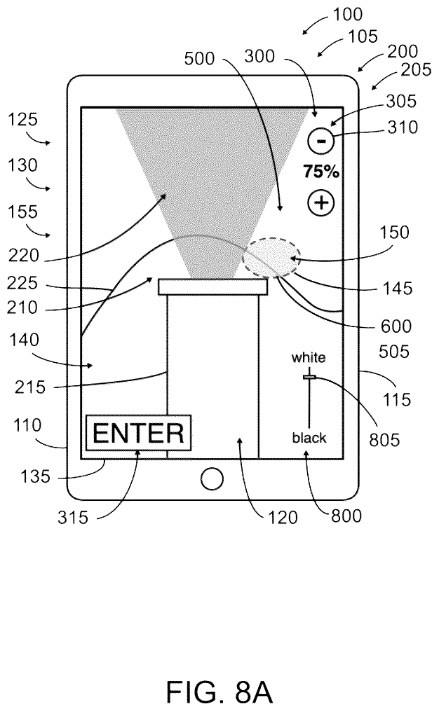

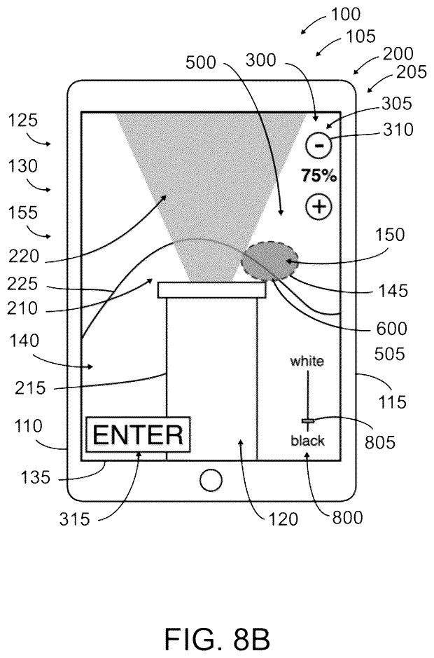

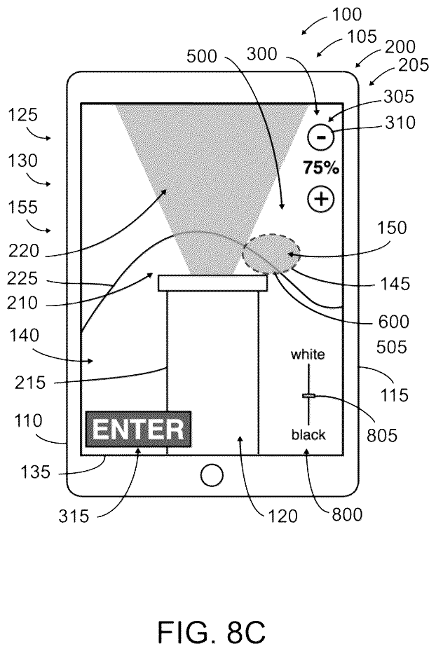

FIGS. 8A, 8B, and 8C show another version of an air opacity determination system 100 similar to the version of FIGS. 6A and 6B, but with the version of FIGS. 8A, 8B, and 8C, the color of the opacity augmentation bubble 600 is adjustable. The color of the smoke 220 that is present in the area of interest 210 can vary. Matching the color of the smoke in the reality image 140 in the augmented reality image 150 makes it easier to determine the opacity of the smoke 220. Accordingly, in this version, the augmented reality adjustment system 300 includes a color adjustment system 800 instead of or in addition to the image positioning system 505 and/or the size adjustment system 700. The color adjustment system 800 in the version of FIGS. 8A, 8B, and 8C is a sliding color scale 805 on the visual image 120 that a user can interact with the adjust the color of the image within the opacity augmentation bubble 600 in a way that most closely matches the color of the smoke 220. In each of FIGS. 8A, 8B, and 8C, the opacity levels are set the same, but FIG. 8A has a color setting too white to match the color of the smoke 220, and FIG. 8B has a color setting too black to match the color of the smoke 220. FIG. 8C shows a properly selected color match which makes the opacity match more able to be easily recognized and determined. In one version, the color of the augmented reality image 150 can be provided using gamma color space, linear color space, or another color space.

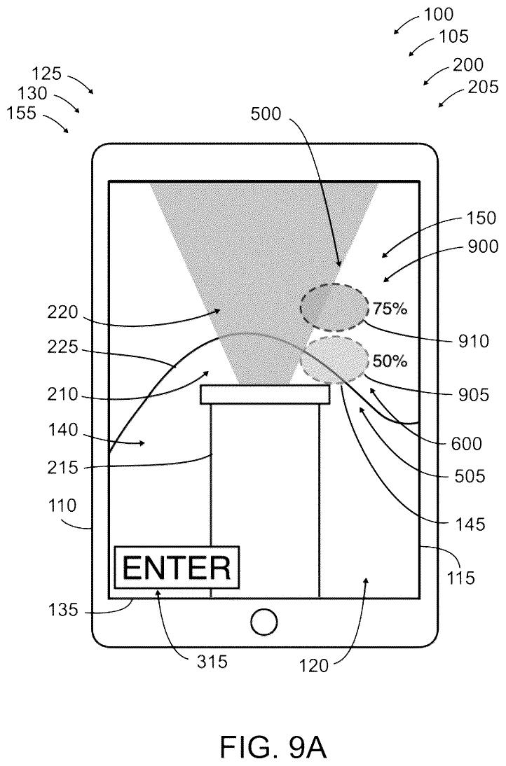

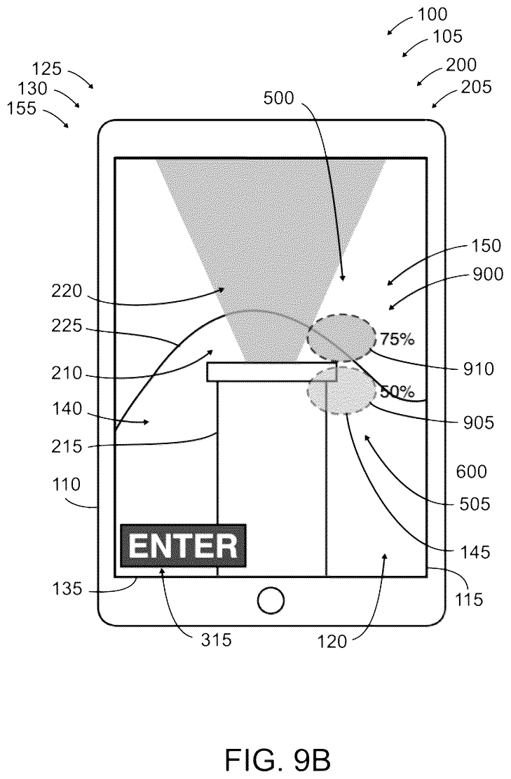

Another version of an air quality assessment system 100 that has an air opacity assessment system 205 utilizing a superimposed display 500 with an augmented reality image 150 in the form of an opacity augmentation bubble 600 superimposed over a reality image 130 is shown in FIGS. 9A and 9B. Whereas in the version of FIGS. 6A and 6B, an assessor adjusts the opacity level of the opacity augmentation bubble 600 to be able to home in on a determined opacity level of the smoke 220 in a reality image 130, the version of FIGS. 9A and 9B allows for simplified side by side comparisons of the reality image 130 with multiple different simulated opacity levels. Accordingly, in the version of FIGS. 9A and 9B, the augmented reality image 150 comprises multiple images with each image being differently augmented. For example, in the particular version of FIGS. 9A and 9B, the augmented reality image 150 comprises an opacity augmentation bubble 600 that is a multi-image opacity augmentation bubble 900. The multi-image opacity augmentation bubble 900 comprises a first bubble component 905 and a second bubble component 910. The first bubble component 905 is positionable at any location over the reality image 140 and is adapted to show what the area within the first bubble component 905 would look like if obscured by a first particular percentage of opacity, such as a first predetermined or entered percentage of opacity. The second bubble component 910 is positionable at any location over the reality image 140 and is adapted to show what the area within the second bubble component 905 would look like if obscured by a second particular percentage of opacity, such as a second predetermined or entered percentage of opacity. The first bubble component 905 and the second bubble component 910 can be moved together and/or separately on the reality image 130 by the image position system 505, as desired.

In the particular version of FIGS. 9A and 9B shown, the multi-image opacity augmentation bubble 900 includes a first bubble component 905 that simulates an opacity level of 50% and a second bubble component that simulates an opacity level of 75%. In FIG. 9A, the first bubble component 905 is positioned in proximity to the position where the opacity of the smoke 220 in the reality image 140 is to be determined and more specifically in a position where the background object 225, such as the hillside and/or the sky, can be most easily compared to the background object 225 in the unaugmented reality image 140. In this position and in this example, it can be seen that the smoke 220 in the reality image 140 is more opaque than the simulation created by the first bubble component 905 simulation. The assessor then positions the augmented reality image 150 so that the second bubble component 910 is positioned in proximity to the smoke 220 and background 225 of the reality image 140. In this second position, a match is determined to exist, and the assessor then enters the determined opacity level, such as by depressing the opacity level enter button 315 or in another manner.

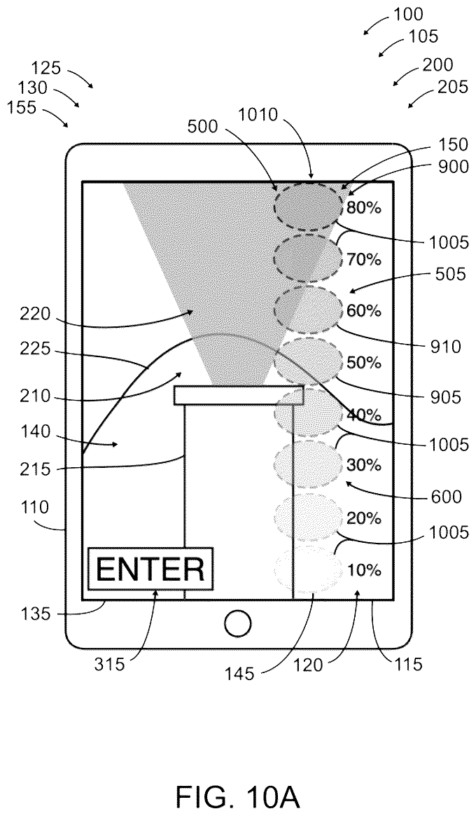

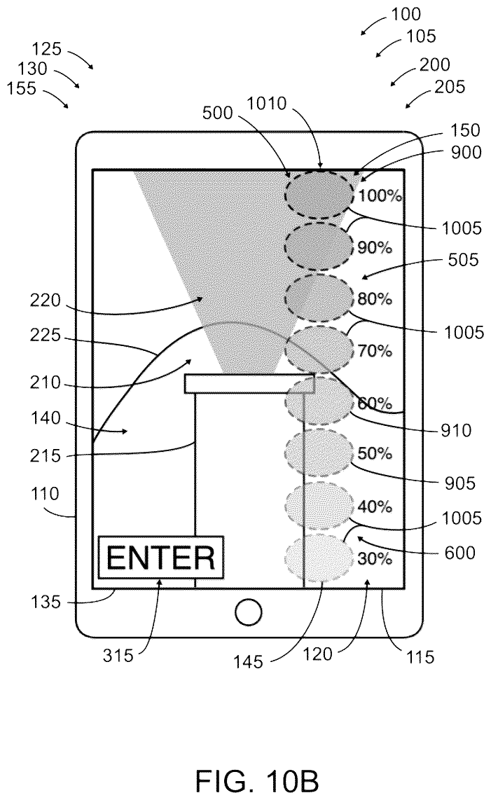

FIGS. 10A and 10B show a version of an air opacity determination system 205 similar to the version of FIGS. 9A and 9B. However, in the version of FIGS. 10A and 10B, the multi-image opacity augmentation bubble 900 includes the first bubble component 905, the second bubble component 910, and one or more additional bubble components 1005. These additional bubble components 1005 allow for more precision to be used during the homing in process of determining the opacity level of the smoke 220 in the reality image 140. As can be seen, in the particular version of FIGS. 10A and 10B, the multi-image opacity augmentation bubble 900 is arranged as a scroll of augmentation bubble components 1010 with each adjacent bubble component changing in simulated opacity by a particular or predetermined amount, such as the 10% increments shown. In one version, the scroll 1010 of augmentation bubble components 1010 can be moved to a desired position on the reality image 140 by the image positioning system 505 and can then be scrolled by the image positioning system 505 so that different bubble components are positioned in proximity to the area of smoke 220 on the reality image 140 that is to be assessed. FIG. 10A shows the scroll of augmentation bubble components 1010 moved to an initial scroll position with a bubble component associated with a 50% opacity level positioned in proximity to the smoke 220 of the reality image 140. In the example shown, the assessor can see that the opacity level of the smoke 220 in the reality image 140 is higher than 50%, and the assessor then can scroll the augmented reality image 150, such as by touching and dragging, so that a bubble component with a closer approximation is positioned in proximity to the smoke 220 of the reality image 140, as shown in FIG. 10B. In the particular example shown, the observed opacity level of the smoke 220 is determined to be somewhere between the 70% bubble component and the 80% bubble component, and the assessor can then draw the conclusion that the opacity level of the smoke 220 is between those two percentages.

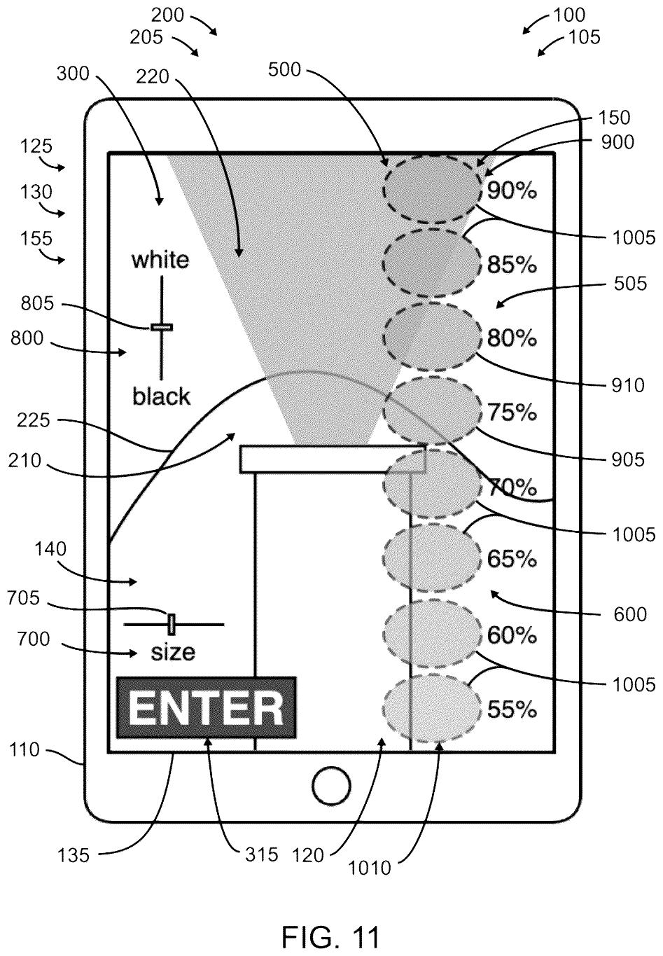

By adding bubble components to the scroll of augmentation bubble components 1010, the opacity level of the smoke 220 in the reality image 140 can be even more pinpointed. For example, FIG. 11 shows a version similar to FIGS. 10A and 10B but with 5% opacity increments between adjacent bubble components. As also shown in the version of FIG. 11, the scroll 1010 of augmentation bubble components 1010 can be sized to a desired size on the reality image 140 by the size adjustment system 700, and/or the color of the simulated smoke in the bubble components can be adjusted by the color adjustment system 800.

In any of the versions disclosed herein, the augmented reality module 155 of the controller 130 can be any system, program, or routine that presents an augmented reality image 150 as described. For example, when being used as an air opacity determination system 205 and when the second image is a portion of the area of interest 210, such as the background object 225, with a simulated particular percentage of opacity thereover, the augmented reality module 155 can be a display system where a graphical layer or a graphical filter is interposed over the reality image so that the portion of the reality image with the interposed graphical layer makes up the augmented reality image. Alternatively, the augmented reality module 155 can receive the electronic image of the area of interest and digitally manipulate a portion thereof to generate the augmented reality image.

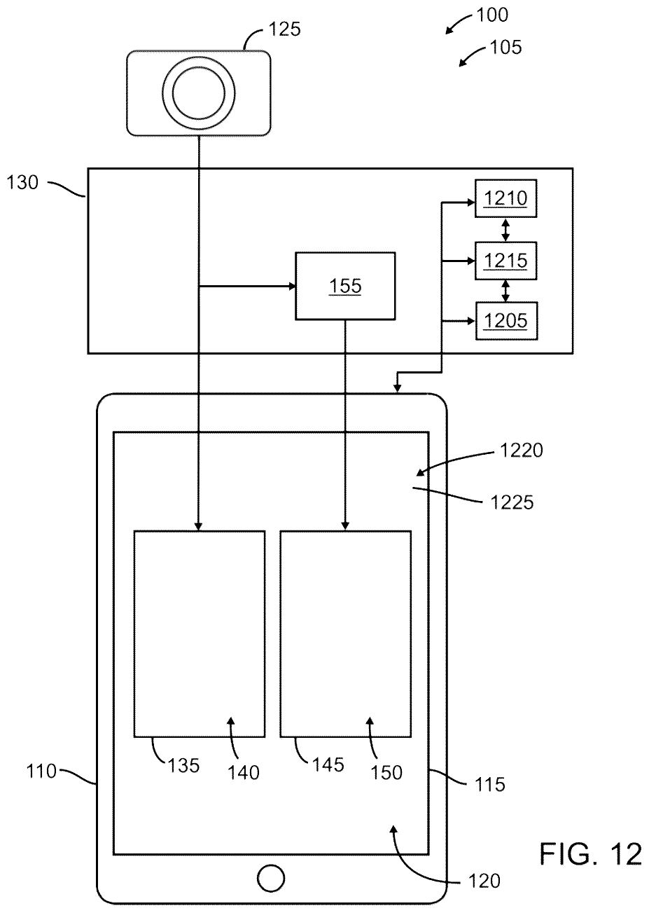

FIG. 12 shows another version of an air quality assessment system 100 which may include or be in the form of an air opacity determination system 205. The version of FIG. 12 is similar to the version of FIG. 1 which is embodied by other figures thereafter all of which may also be embodiments of the version of FIG. 12. In the version of FIG. 12, the controller 130 includes one or more additional modules, such as an input module 1205, a post-processing module 1210, and/or a storage module 1215. The input module 1205, the post-processing module 1210, and/or the storage module 1214 may be in communication with one another and/or with the display device 110. The input module 1205 may be provided so that the controller 130 may be capable of interacting with a user of the air quality assessment system 100 via an input device 1220, such as a keyboard, a graphical user interface, wireless communication, voice command, or any other manner. In the version shown in FIG. 12, the input device can be a graphical user interface provided on a touchscreen 1225 of the display device 110. For example, the user can information into input module 1205 that can be used to control or manage the air opacity determination system 205 in use, such as by entering information related to the augmented reality adjustment system 300 including the image positioning system 505, the size adjustment system 700, the color adjustment system 800, and/or the like. Alternatively or in addition, the input module 1205 can receive input data entered by the user relevant to the opacity determination including one or more of the direction from the camera 125 to the area of interest 210 where there is visible smoke to be assessed, the viewing angle between user or the camera 125 and the area of interest 210, the name of a facility being assessed, process equipment information, control device information, the position of the sun relative to the camera 125 and/or the area of interest, the amount of cloud cover or other weather information, such as temperature, humidity, wind speed, wind direction, wet bulb temperature, or the like, the test duration, the observation frequency, pictures and/or videos of the visible emissions and surroundings, pauses during the assessment and reasons therefore, assessment comments, device make and model, camera specifications, and/or the like. Any such information or data entered into the input module 1205 can be used by the air quality assessment system 100 during a test process and/or can be stored by the storage module 1215 for later review. The post-processing module 1210 can manage the assessment information following an assessment process. For example, assessment data and results can be stored locally on internal storage or external storage within the storage module 1215 and/or the assessment data and results can be uploaded and synced with a user's account on a remote server or cloud storage platform. Data on the remote server can be manipulated and additional fields may be present for manual data entry. Manually entered data may include, for example, one or more of facility permit number, facility address, facility contact name, facility contact phone number, observer or assessor's name, certification date and issuer, and/or company, a plume shape, a visible emission type, an emission color, a presence of water vapor plume, an averaging period, and/or the like. Data from the server can be compiled into a test report and printed or saved.

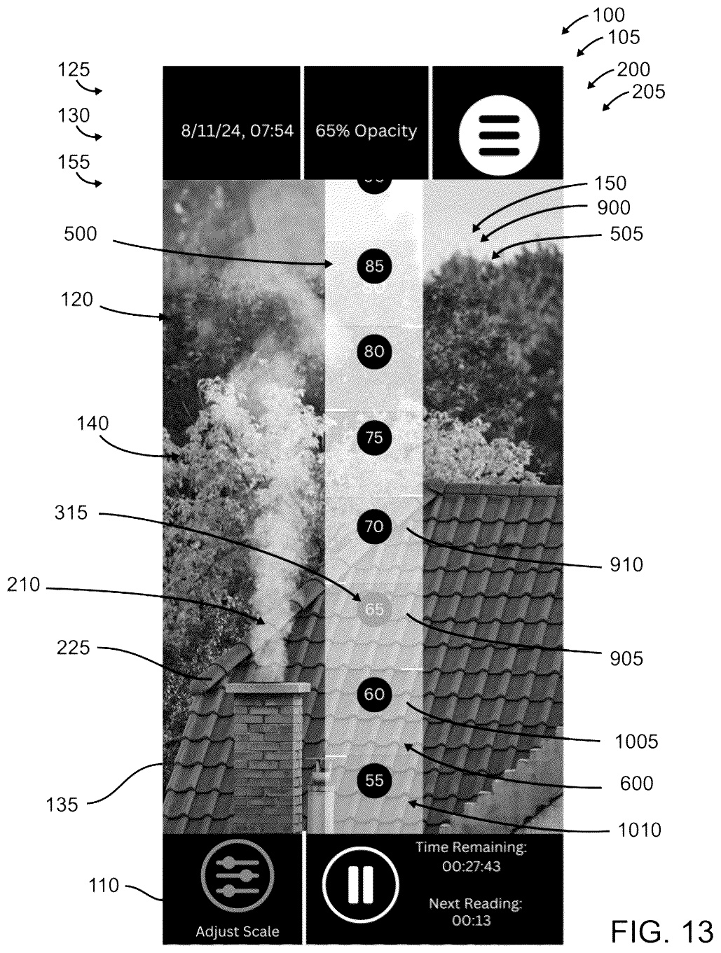

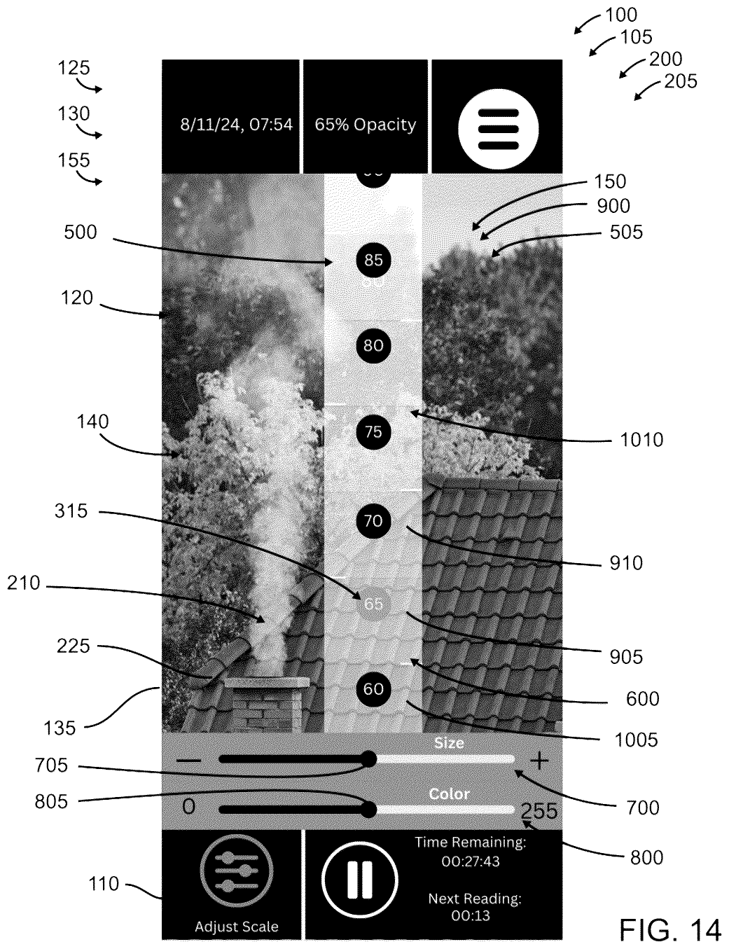

In one particular version, the air quality assessment system 100 is in the form of an air opacity determination system 205 that is specifically designed and configured to an EPA Method 9 determination. FIGS. 13 and 14 illustrate screen shots of a smart phone or tablet app designed for this purpose. Additional screen shots showing additional features of a particular version of an app designed for this purpose are shown in Appendix A and associated description in U.S. Provisional Patent Application 63/706,839 filed on Oct. 14, 2024, which is incorporated herein by reference.

The air quality assessment system 100, and particularly in the form of an air opacity determination system 205 designed to aid in Method 9 determinations, solves or improves upon many of the problems of the conventional systems. For example, the air quality assessment system 100 improves one or more of (i) accuracy, since opacity observations are compared to a reference tool and are not subject to the memory of an assessor, (ii) evidence creation, since the opacity observations can be accompanied with a screenshot of the plume and the augmented reality reference image and/or other information, (iii) convenience, since assessors may not need to attend smoke school or in-person training, or at least attend it as often, (iv) affordability, since the cost to train and certify is significantly lower than in-person training due to decreased time and travel expenses and the fact that there are fewer special hardware requirements, (v) repeatability, since opacity observations are more consistent and repeatable and less dependent on the assessor or the assessor's memory, and (vi) accountability, since the system can prevent opacity readings that are intentionally too high or low by including a record of the visual images. Due to these factors, facilities and organizations can have more trained staff and ensure better compliance for less cost than existing methods. Because the air quality assessment system 100 is a tool that can be used by anyone with a phone or tablet, it opens the door to new markets that have been unserved or underserved. A smoke generator is no longer needed, or at least as needed, and special equipment is no longer required, or at least as needed.

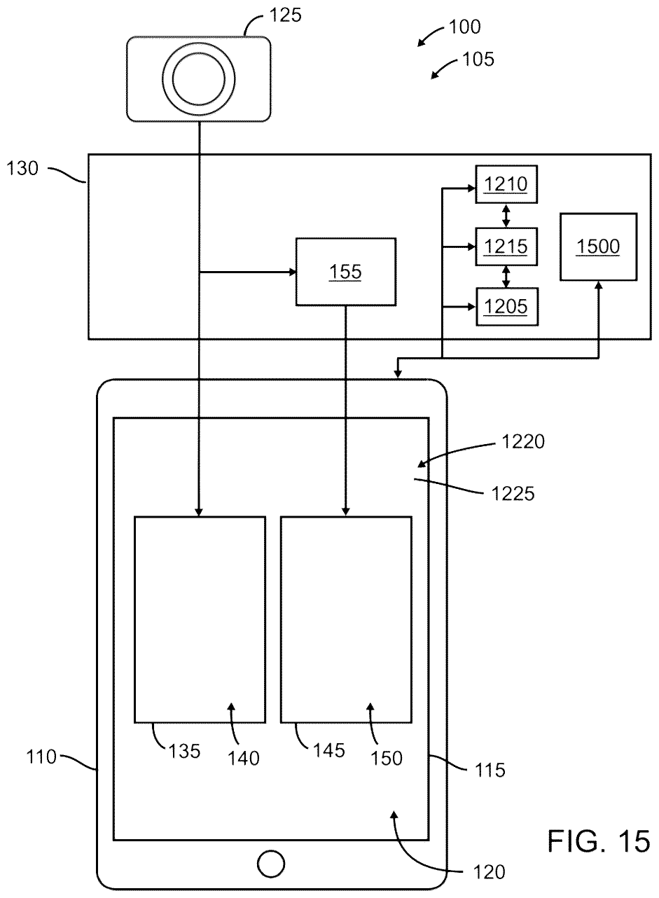

FIG. 15 shows another version of an air quality assessment system 100 which may include or be in the form of an air opacity determination system 205. The version of FIG. 15 is similar to the version of FIGS. 1 and/or 12 which are embodied by other figures shown or discussed herein, all of which may also be embodiments of the version of FIG. 15. In the version of FIG. 15, the controller 130 includes an artificial intelligence module 1500. The artificial intelligence module 1500 includes code and/or routines that allow for artificial intelligence or machine learning processes to be run on the data from air quality assessment processes. For example, captured reality images 140 and/or augmented images 150 can be used to train the artificial intelligence module 1500. Once trained, the artificial intelligence module 1500 can make predictive pre-determinations of an air quality, such as an air opacity level, to aid a human assessor and/or can make automatic determinations of opacity levels that can, optionally, be confirmed by a certified user. Artificial intelligence systems, such as the one included within the artificial intelligence module 1500, are particularly accurate and fast when comparing specific features of compared images. Accordingly, by allowing the artificial intelligent module 1600 to compare the air opacity in the reality image 140 with various augmented reality images 150 of varying opacities, the trained artificial intelligence module 1500 can provide reliable results in minimal time. The results from the artificial intelligence module 1500 can be displayed by the visual display 120, stored in the storage module 1215, and/or included for use by the post-processing module 1210.

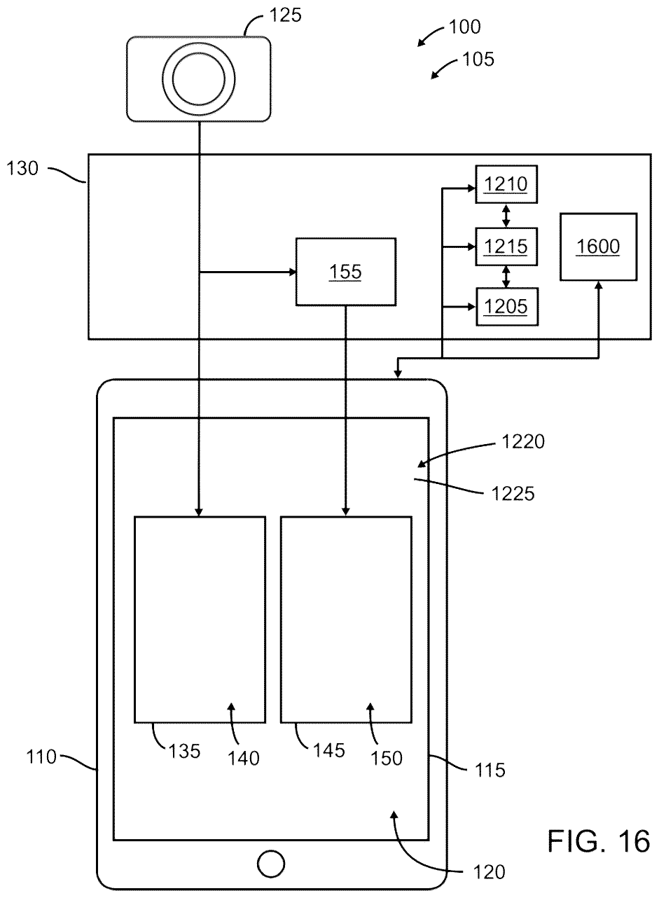

FIG. 16 shows another version of an air quality assessment system 100 which may include or be in the form of an air opacity determination system 205. The version of FIG. 16 is similar to the version of FIGS. 1, 12, and/or 15 which is embodied by other figures thereafter all of which may also be embodiments of the version of FIG. 16. In the version of FIG. 16, the controller 130 includes a training module 1500. The training module 1600 can include routines that allow a user to train to be an assessor and/or that can be an aid to a user who is training to be an assessor, such as an individual getting certified or re-certified to make determination under EPA Method 9. An assessor being trained may compare images or videos that contain real or simulated areas of interest 210 with images of known opacity, and the assessor may demonstrate proficiency of the application by accurately assigning the opacity value. In one version, the training module 1600 includes a testing module or certification module that can be used to test or determine a user's ability to make accurate determinations of air opacity and can optionally be used to certify a user under programs such as EPA Method 9 or the like. For example, the air quality assessment system 100 can be used to certify or re-certify an individual under an air opacity assessment method, such as EPA Method 9 by displaying a reality image 140, which may be a real or simulated reality image and displaying an augmented reality image 150 of a portion of the reality image at a particular opacity in juxtaposition to the reality image 140, and tasking the individual to compare the reality image 140 to the augmented reality image 150 to make a determination of the air opacity of the reality image. The determination can then be assessed for accuracy and a decision can be made as to whether or not the individual should be certified or not or re-certified or not.

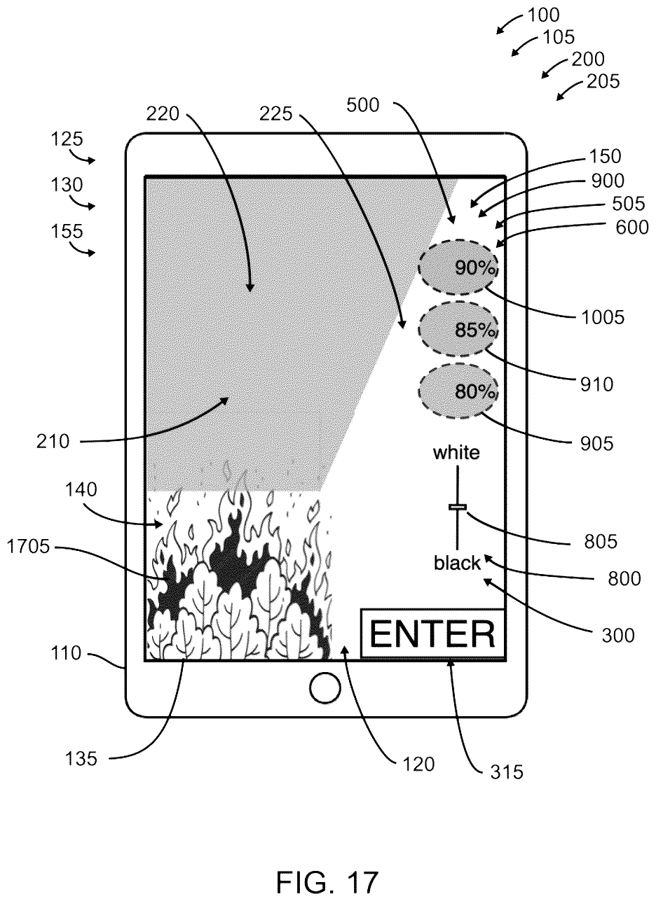

While the air quality assessment system 100 has been exemplified above in the context of being useful for determining opacity of smoke 220 emitted from a smokestack 215 or the like, the air quality assessment system 100 has numerous other uses. For example, FIG. 17 shows another application or use for the air quality assessment system 100 in the form of an air opacity determination system 205. The version of FIG. 17 is similar to the version of FIGS. 9A and 9B but can be likewise adapted be similar to any of the versions described herein. In the version of FIG. 17, the smoke 220 that is being assessed is smoke emitted from a wildfire 1705. The opacity and/or color of the smoke 220 can provide useful information concerning the conditions of the wildfire and/or the concentrations of smoke at particular distances from the wildfire 1705. Another optional use for the air quality assessment system 100 in the form of an air opacity determination system 205 includes an assessment of fog or smog opacity and/or color. Another potential use of the air opacity determination system 205 includes the assessment of tint level in a window or glass.

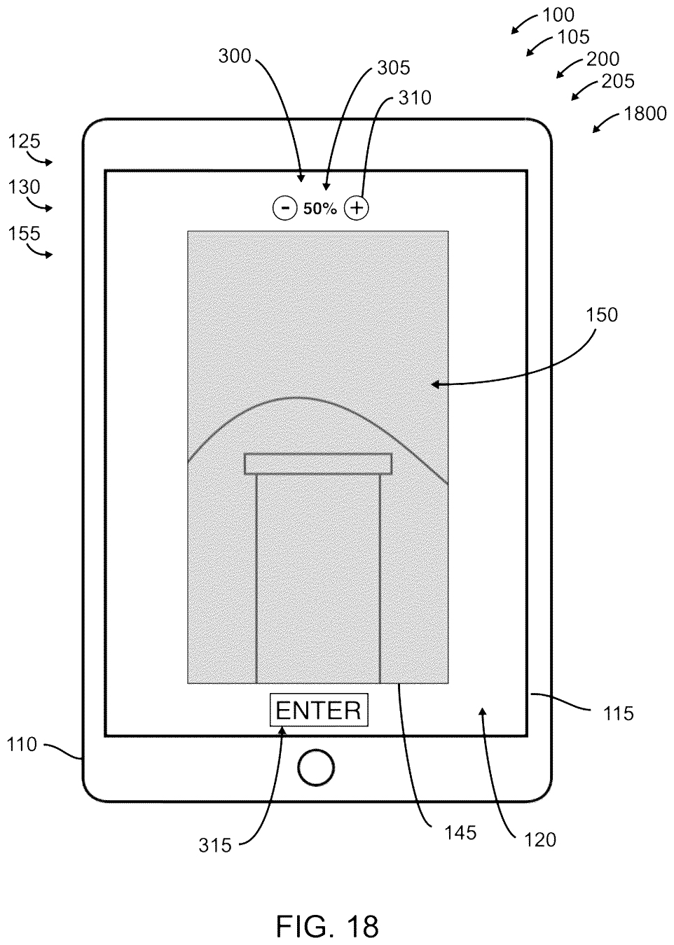

In the versions shown and described heretofore, the air quality assessment system 100 displays a reality image 140 and an augmented reality image 150 juxtaposed with respect to one another to aid an assessor in making a determination of air opacity or other air quality assessment. The juxtaposition can be a side-by-side arrangement, an overlapping arrangement, a superposition, and/or the like. FIG. 18 shows another application or use for the air quality assessment system 100 in the form of an air opacity determination system 205. The version of FIG. 18 is similar to the version of FIGS. 3A, 3B, and 3C, or any other version described herein, but the version of FIG. 18 is an augmented reality image only display version 1800 where the augmented reality image 150 is displayed on the visual image 120 without a corresponding reality image. In using this version, the assessor would view the area of interest live and with their own eyes rather than looking at a reality image of the area of interest. The assessor will compare the real-world observation to the reference augmented reality image 150 to make the opacity determination. When the version of FIG. 18 is embodied in a smart phone, tablet, or the like, the assessor can optionally hold the display device 110 in a position and orientation where it is in juxtaposition to the area of interest being observed. A reality image may later be added for keeping recorded evidence.

Examples of screenshots of a particular smart phone app version of an air opacity determination system 205 and accompanying features is shown in the Appendix of U.S. Provisional Patent Application 63/706,839 filed on Oct. 14, 2024, and the Appendix and its description are incorporated herein by reference.

Although the present invention has been described in considerable detail with regard to certain preferred versions thereof, other versions are possible, and alterations, permutations and equivalents of the versions shown will become apparent to those skilled in the art upon a reading of the specification and study of the drawings. For example, the cooperating components may be reversed or provided in additional or fewer number, and all directional limitations, such as up and down and the like, can be switched, reversed, or changed as long as doing so is not prohibited by the language herein with regard to a particular version of the invention. Like numerals represent like parts from figure to figure. When the same reference number has been used in multiple figures, the discussion associated with that reference number in one figure is intended to be applicable to the additional figure(s) in which it is used, so long as doing so is not prohibited by explicit language with reference to one of the figures. Also, the various features of the versions herein can be combined in various ways to provide additional versions of the present invention. Furthermore, certain terminology has been used for the purposes of descriptive clarity, and not to limit the present invention. Throughout this specification and any claims appended hereto, unless the context makes it clear otherwise, the term “comprise” and its variations such as “comprises” and “comprising” should be understood to imply the inclusion of a stated element, limitation, or step but not the exclusion of any other elements, limitations, or steps. Throughout this specification and any claims appended hereto, unless the context makes it clear otherwise, the term “consisting of” and “consisting essentially of” should be understood to imply the inclusion of a stated element, limitation, or step and the exclusion of any other elements, limitations, or steps or the exclusion of any other essential elements, limitations, or steps, respectively. Throughout the specification, any discussion of a combination of elements, limitations, or steps should be understood to include (i) each element, limitation, or step of the combination alone, (ii) each element, limitation, or step of the combination with any one or more other element, limitation, or step of the combination, (iii) an inclusion of additional elements, limitations, or steps (i.e. the combination may comprise one or more additional elements, limitations, or steps), and/or (iv) an exclusion of additional elements, limitations, or steps or an exclusion of essential additional elements, limitations, or steps (i.e. the combination may consist of or consist essentially of the disclosed combination or parts of the combination). All numerical values, unless otherwise made clear in the disclosure or prosecution, include either the exact value or approximations in the vicinity of the stated numerical values, such as for example about +/−ten percent or as would be recognized by a person or ordinary skill in the art in the disclosed context. The same is true for the use of the terms such as about, substantially, and the like. Also, for any numerical ranges given, unless otherwise made clear in the disclosure, during prosecution, or by being explicitly set forth in a claim, the ranges include either the exact range or approximations in the vicinity of the values at one or both of the ends of the range. When multiple ranges are provided, the disclosed ranges are intended to include any combinations of ends of the ranges with one another and to include zero and infinity as possible ends of the ranges. Therefore, any appended or later filed claims should not be limited to the description of the preferred versions contained herein and should include all such alterations, permutations, and equivalents as fall within the true spirit and scope of the present invention.

What is claimed is:

1. An air quality assessment system to assist an assessor in making an air opacity determination, the system comprising:
a computer device comprising a display adapted to display a visual image, a camera adapted to capture an electronic image of an area of interest, and a controller in communication with the camera and the display, the controller capable of generating an augmented reality version of the electronic image of the area of interest,
wherein the visual image displayed by the display includes a first image and a second image,
wherein the first image comprises a reality image of the area of interest,
wherein the second image comprises an augmented reality image of the area of interest, wherein the augmented reality image shows at least a portion of the area of interest at a particular percentage of opacity, and
wherein an assessor can compare the reality image to the augmented reality image to aid the assessor in making an air opacity determination of the reality image.

2. A system according to claim 1 wherein the electronic image of the area of interest includes smoke and a background object and wherein the augmented reality image shows what at least a portion of the background object would look like if smoke having the particular percentage of air opacity were between the camera and the background object.

3. A system according to claim 1 wherein the color of smoke in the augmented reality image is adjustable.

4. A system according to claim 1 wherein the particular percentage of opacity in the augmented reality image is adjustable.

5. A system according to claim 1 wherein the size or position of the augmented reality image is adjustable relative to the first image.

6. A system according to claim 1 wherein a screen shot can be taken and stored showing the reality image and the augmented reality image.

7. A system according to claim 1 wherein the first image and the second image overlap so that the augmented reality image is superimposed over at least a portion of the reality image.

8. A system according to claim 1 wherein the augmented reality image is in the form of an opacity augmentation bubble that can be positioned over a portion of the reality image to show the portion of the reality image at the particular percentage of opacity.

9. A system according to claim 1 wherein the augmented reality image comprises a first augmented reality image and a second augmented reality image, wherein the first augmented reality image and the second augmented reality image are differently augmented.

10. A system according to claim 9 wherein the first augmented reality image shows at least a portion of the area of interest with a first particular percentage of opacity, wherein the second augmented reality image shows at least a portion of the area of interest with a second particular percentage of opacity, and wherein the first particular percentage of opacity and the second particular percentage of opacity are different.

11. A system according to claim 9 wherein the augmented reality image comprises a scroll of multiple opacity augmentation bubbles that can each be positioned over a portion of the reality image to show what the portion of the reality image would look like at the particular percentage of opacity, wherein each opacity augmentation bubble has a different particular percentage of opacity.

12. A system according to claim 1 wherein the reality image is a photographic image or a video image, and wherein the reality image is a live image or a recorded image.

13. A system according to claim 1 wherein the controller comprises a training module, the training module including routines that allow a user to practice making air opacity determinations of a recorded reality image.

14. A system according to claim 1 wherein the controller comprises a certification module, the certification module including routines that allow a user to demonstrate the user's ability to make air opacity determinations so the user can be certified or re-certified under an air opacity assessment method.

15. A system according to claim 1 wherein the air quality assessment system comprises a downloadable application running on an electronic device, wherein the electronic device includes the camera and display.

16. A method of assessing air quality by determining air opacity, the method comprising:
providing an air quality assessment system comprising a computer device comprising a display adapted to display a visual image and a camera adapted to capture an electronic image of an area of interest;
capturing an electronic image of the area of interest;
displaying the electronic image as a first image, wherein the first image is a reality image of the area of interest;
displaying a second image, wherein the second image is an augmented reality image, wherein the augmented reality image shows at least a portion of the area of interest at a particular percentage of opacity;
comparing the reality image to the augmented reality image; and
making a determination of the air opacity of the reality image.

17. A method according to claim 16 wherein the first image and the second image are juxtaposed with respect to one another.

18. A method according to claim 16 further comprises adjusting one or more of the size, position, color, and augmentation of the second image relative to the first image.

19. A method according to claim 16 wherein the second image comprises multiple images, wherein each of the multiple images has a different particular percentage of opacity.

20. A method of assessing air quality by determining air opacity, the method comprising:
providing an air quality assessment system comprising a computer device comprising a display adapted to display a visual image and a camera adapted to capture an electronic image of an area of interest; and
using the air quality assessment system to certify or re-certify an individual under an air opacity assessment method by:
displaying the electronic image of an area of interest as a first image, wherein the first image is a reality image of the area of interest;
displaying a second image, wherein the second image is an augmented reality image, wherein the augmented reality image shows at least a portion of the area of interest at a particular percentage of opacity;
tasking the individual to compare the reality image to the augmented reality image to make a determination of the air opacity of the reality image; and
assessing the accuracy of the determination.
